(19) United States
(12) Patent Application Publication
(10) Pub. No.: US 2023/0364338 A1
(43) Pub. Date: Nov. 16, 2023

(54) MULTIPLE TUBE INTRAVENOUS FLUID DELIVERY SYSTEM

(71) Applicant: Dukal, LLC, Ronkonkoma, NY (US)

(72) Inventors: Christopher PAGE, Setauket, NY (US); Donald Lee HANNULA, San Luis Obispo, CA (US); Michael R. BIELSKI, Manorville, NY (US)

(21) Appl. No.: 18/196,249

(22) Filed: May 11, 2023

Related U.S. Application Data
(60) Provisional application No. 63/340,763, filed on May 11, 2022.

(51) Int. Cl. A61M 5/168; A61M 39/24

(52) U.S. Cl. CPC A61M 5/16827; A61M 39/24

(57) ABSTRACT

An intravenous fluid delivery system includes a multiple tube portion including multiple IV fluid lines carrying fluid that are removably connected to each other and connectable to separate IV sources and a mixing element connected to the multiple IV fluid lines and configured to mix the fluid from the multiple IV fluid lines and provide them to a single outlet.

### CROSS-REFERENCE TO RELATED APPLICATIONS

The present application claims benefit of and priority to U.S. Provisional Patent Application Ser. No. 63/340,763 filed May 11, 2022 entitled MULTIPLE TUBE INTRAVENOUS FLUID DELIVERY SYSTEM, the entire content of which is hereby incorporated by reference herein.

### FIELD OF THE DISCLOSURE

The present invention relates to an intravenous (IV) fluid delivery system and specifically an intravenous fluid delivery system including multiple lines and a mixing element configured to combine fluid from the multiple lines to provide a single output to provide intravenous fluid to a patient.

### RELATED ART

In various environments, including industrial, laboratory and medical settings, combining different fluids, including chemicals, medications, and other fluids, to name a few, subject to strict sequence and timing parameters may be critical to success. In many applications, such materials must be separated at certain times and then combined at certain times to be effective. In one example, the timing of adding water to cement or concrete may be critical to ensure that the material will not dry before use and/or to ensure that it has a proper consistency for application. In another example, laboratory research may require that chemicals be added and removed at precise times to optimize reactions and avoid degradation of materials.

In medicine, particularly in operating rooms, recovery rooms and intensive care units, the concurrent delivery of multiple medications as well as nutrients and other fluids to a patient via intravenous (IV) infusion is common. In many cases, it may be important or expedient to keep the materials separate at some points in time and to combine them at others. In addition, it may be important to quickly change fluids and or to adjust rates differently for different materials. Indeed, the ability to efficiently alter administration of these substances may have profound implications on their efficacy and the safety of the patient.

In conventional IV tubing systems, dose and rate changes may be implemented via multiple separate sets of IV tubing connected at a distance from the patient to IV fluid/medication bags and/or infusion pumps or other sources. When multiple medications and/or other fluids are to be administered through fewer IV access points on the patient, stopcocks are often used to allow multiple lines and supply bags to be fed into a single carrier line, sometimes at a significant distance from the patient.

These conventional systems have inherent flaws that may result in errors as well as inefficiency that may compromise the safety of the patient. The use of multiple IV lines poses logistical challenges since it is important to avoid tangles, knots and/or inadvertent disconnection of the IV lines. Where such tangles, knots or disconnections occur, it is often necessary to disconnect and reconnect some or all of the tubes involved which increases the risk of infection and errors. In some cases, the connection of the wrong tube to a patient may be extremely dangerous and may be fatal in some cases.

In addition, feeding multiple lines into a carrier line at a distance from the patient's body may result in a delay in administration of the medicine or other fluid being provided. In some cases, boluses of fluid or medication may accumulate in the main carrier line for a period of time and result in excessive administration of fluid when they finally enter the patient's vein.

Accordingly, it would be beneficial to provide an intravenous fluid delivery system that avoids these and other problems.

### SUMMARY

It is an object of the present disclosure to provide a multiple line intravenous fluid delivery system suitable for providing multiple intravenous fluids to a patient via a single intravenous needle or catheter.

It is a further object of the present disclosure to provide a fluid delivery system that allows greater control and facilitates precise mixing of chemicals, fluids, medications, and other materials at a point close to the patient and simplifies administration of multiple medications and fluids.

An intravenous fluid delivery system in accordance with an embodiment of the present disclosure includes a multiple line portion, the multiple line portion including a plurality of fluid lines configured to transport fluid, wherein the plurality of fluid lines are connected to each other; and a mixing element provided at a distal end of the multiple line portion wherein each fluid line of the plurality of fluid lines is in fluid communication with the mixing element, the mixing element including a single outlet in fluid communication with each fluid line of the plurality of fluid lines such that fluid from each of the fluid lines flows through the single outlet.

In embodiments, the plurality of fluid lines includes a central line and one or more peripheral lines positioned around the central line.

In embodiments, the one or more peripheral lines are connected to an external wall of the central line.

In embodiments, the one or more peripheral lines are detachably connected to the central line along at least a portion of a length of the one or more peripheral lines and the central line.

In embodiments, the one or more peripheral lines are separable from the central line along at least a portion of the one or more peripheral lines and the central line.

In embodiments, a thickness of a connection between the central line and the one or more peripheral lines is between 0.005 in and 0.030 in.

In embodiments, the plurality of fluid lines are made of PVC, silicone, Polyurethane or Tygon.

In embodiments, the plurality of fluid lines are extruded together.

In embodiments, the system includes a plurality of connectors, wherein each connector of the plurality of connectors is configured for connection to a respective fluid source and a respective fluid line such that each of the fluid lines is connected to a respective fluid source.

In embodiments, the respective fluid source is an intravenous bag.

In embodiments, the respective fluid source is an infusion pump.

In embodiments, the mixing element includes a plurality of slots formed in an interior thereof, wherein the plurality of slots correspond to and are in fluid communication with the plurality of fluid lines.

In embodiments, the mixing element includes a central slot corresponding to and in fluid communication with the central line and one or more peripheral slots corresponding to and in fluid communication with the one or more peripheral fluid lines.

In embodiments, the one or more peripheral slots are configured to provide fluid to the central slot.

In embodiments, the single outlet is formed by a distal end of the central slot such that fluid passing through the central slot, including fluid provided to the central slot from the one more peripheral slots passes through the single outlet.

In embodiments, the system includes an outlet tube configured to be received in and in fluid communication with the single outlet.

In embodiments, the system includes a second connector configured to connect the outlet tube to a catheter.

In embodiments, the system includes a second connector configured to connect the outlet tube to an intravenous needle.

In embodiments, the system includes a one-way valve provided downstream from the outlet tube and configured to prevent fluid from flowing upstream into the mixing element.

In embodiments, the system includes a one-way valve positioned between the outlet tube and the mixing element and configured to prevent fluid from flowing upstream into the mixing element.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates a fluid delivery system including multiple lines which may be connected to multiple fluid sources in accordance with an exemplary embodiment of the present disclosure;

FIG. 2 is a more detailed view of a distal end of the fluid delivery system of FIG. 1;

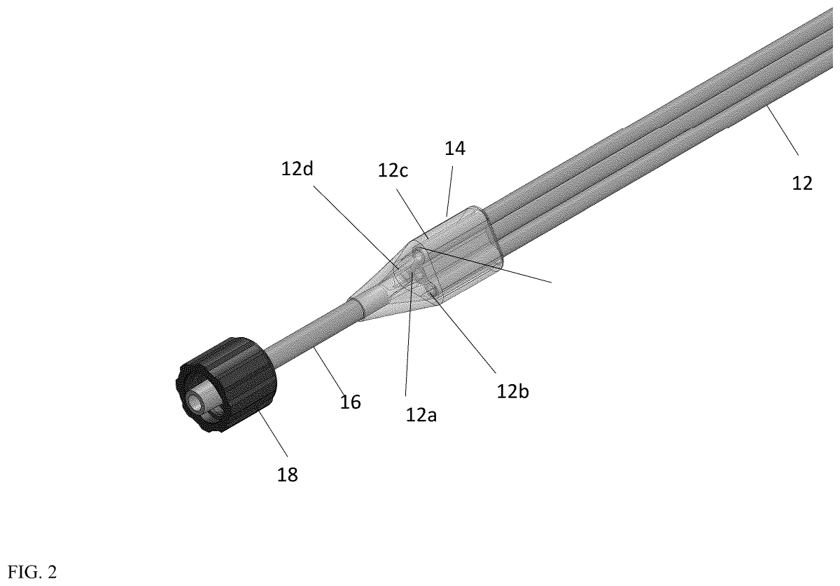

FIG. 3 is an exploded view of the distal end of the fluid delivery system of FIGS. 1 and 2;

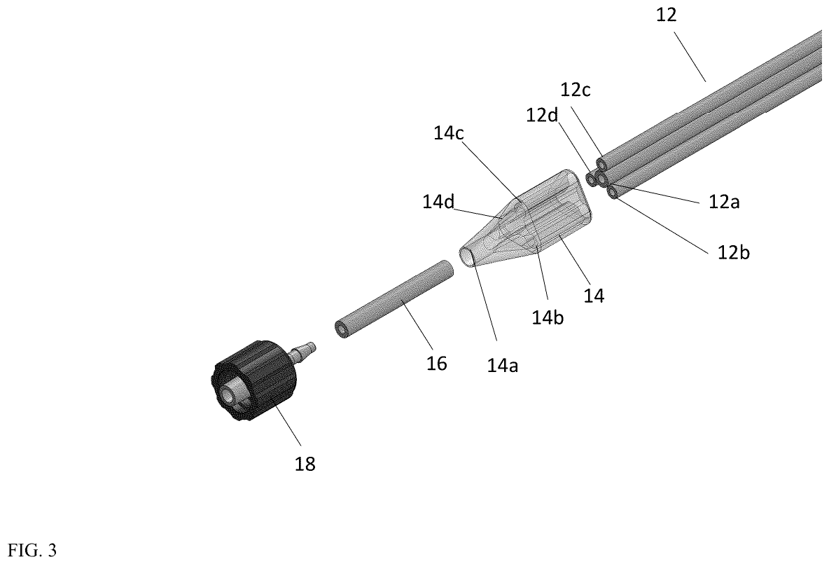

FIG. 4 is a detailed view of a mixing element provided on the distal end of the fluid delivery system to mix fluid from the multiple lines; and

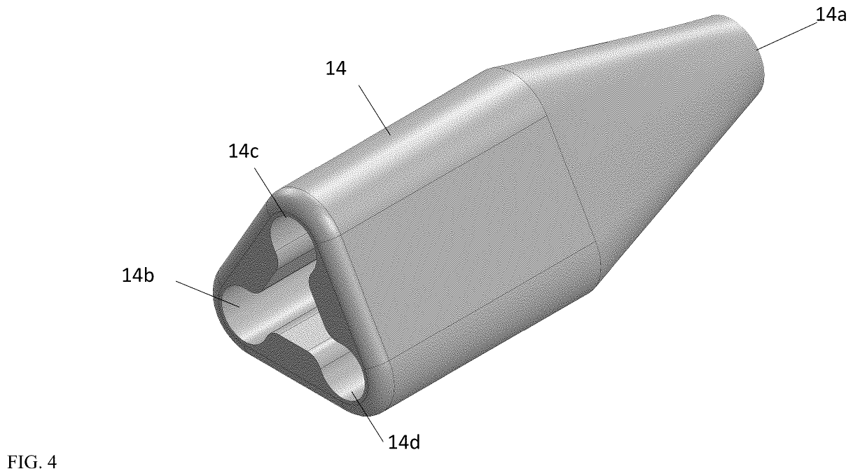

FIG. 5 is a cross-sectional view of the mixing element of FIG. 4.

### DETAILED DESCRIPTION OF THE EXEMPLARY EMBODIMENTS

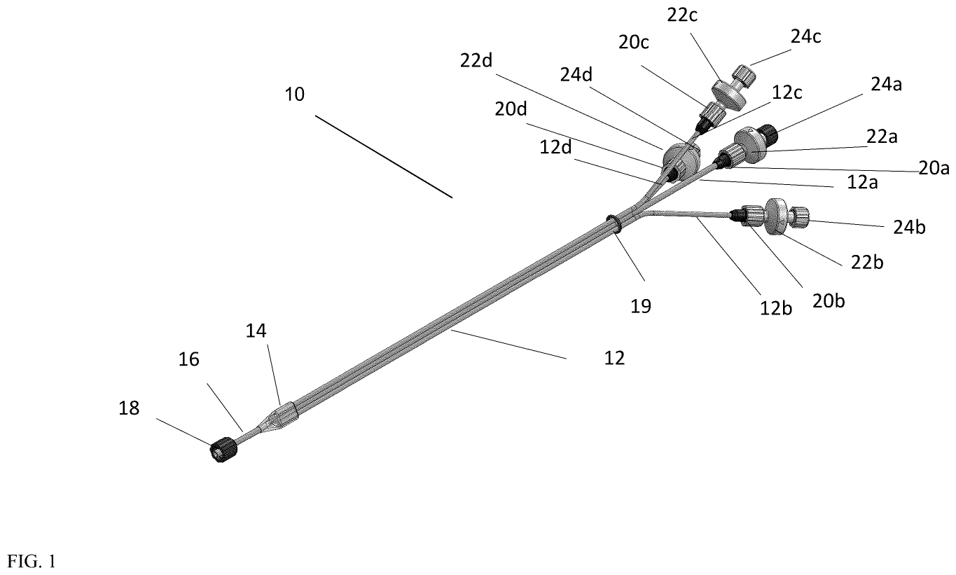

An intravenous fluid delivery system 10 in accordance with an embodiment of the present disclosure is illustrated in FIG. 1. In embodiments, the system 10 includes a multiple line portion 12 including multiple lines 12a, 12b, 12c and 12d configured for connection to separate intravenous fluid supplies, such as IV bags. In embodiments, the multiple lines 12a, 12b, 12c and 12d may be connected to separate infusion pumps. In embodiments, the intravenous fluid supplies, bags or infusion pumps may provide medication, nutrients or any other intravenous fluid. In embodiments, one or more of the lines 12a, 12b, 12c and 12d may be connected to an infusion pump or pumps. While four lines 12a, 12b, 12c and 12d are illustrated, the system 10 may include more lines or fewer lines. In embodiments, the multiple lines are configured with a central line 12a surrounded by peripheral lines 12b, 12c and 12d. In embodiments, other configurations of the lines 12a, 12b, 12c, 12d may be used. In embodiments, all of the fluid lines 12a, 12b, 12c, 12d include a central passage through which fluid flows.

In embodiments, an outer wall of each of the peripheral lines 12b, 12c, 12d may be connected to a central outer wall of the central line 12a. In embodiments, the multiple line portion 12 of the system 10 may be extruded as a single piece during manufacture. In embodiments, the respective connection between each of the peripheral lines 12b, 12c and 12d and the central line 12a may be configured such that the peripheral lines 12b, 12c, 12d may be separated from the central line 12a along at least a portion of their length, as illustrated on the proximal end of the multiple line portion 12 in FIG. 1. In embodiments, the multiple line portion 12, including each of the fluid lines 12a, 12b, 12c, 12d, may be made of a flexible tubing material, which may be PVC, silicone, Polyurethane or Tygon, to name a few. In embodiments, other materials may be used including blends of materials. In embodiments, an O-ring or other binding element 19 may be provided on the multiple line portion 12 to hold the multiple lines 12a, 12b, 12c and 12d together and prevent unwanted separation. In embodiments, this binding element 19 may be moved up and down the multiple line portion 12 to allow for more or less of the length of the respective lines 12a, 12b, 12c and 12d to be separated from each other.

In embodiments, each of the lines 12a, 12b, 12c and 12d may include a female luer lock 20a, 20b, 20c, 20d respectively provided at a top, or proximal, end of each respective line and mating with the tubing of the respective line. In embodiments, any suitable connector or connectors may be provided at a top end of each respective line. In embodiments, each respective female luer lock 20a, 20b, 20c, 20d, or other connector, may be connected to a respective check valve 22a, 22b, 22c, 22d which may be used to control flow of fluid into each line. In embodiments, other valve elements may be used to control flow of fluid into each line. In embodiments, no valve may be provided. In embodiments, a respective cap 24a, 24b, 24c, 24d may be provided upstream of each check valve 22a, 22b, 22c and 22d, or other flow control element, which may be removed to connect each line to an IV fluid supply or infusion pump. In embodiments, the IV fluid supply may be a bag or other container including fluid for intravenous administration to a patient such as saline, medicine, nutrients and any other intravenous fluid, to name a few.

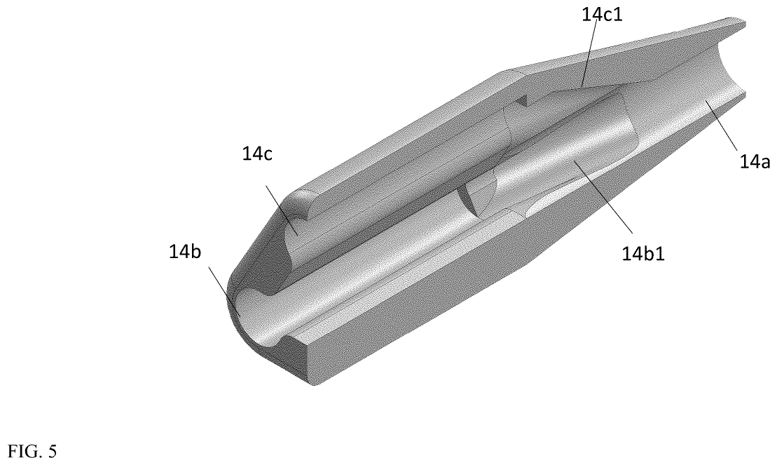

In embodiments, a mixing element 14 may be provided at a distal end of the multiple line portion 12, opposite the top end of the lines. In embodiments, the mixing element 14 may include multiple slots 14b, 14c, 14d configured to receive the distal ends of the peripheral lines 12b, 12c and 12d and a central passage 14a aligned with and receiving the central line 12a. In embodiments, the distal end of each of the respective slots 14b, 14c and 14d may be angled (see elements 14b1, 14c1, for example of FIG. 5) toward the central passage 14a such that fluid provided from the peripheral lines 12b, 12c, 12d passes through the slots 14b, 14c and 14d and is mixed with the fluid provided via the central line 12a in the central passage 14a. In embodiments, where more or fewer lines are used, the mixing element 14 may have more or fewer slots to receive the lines. In embodiments, a single outlet tube 16 may be connected to the distal end of the central passage 14a such that all fluid exits the mixing element 14 through the same outlet. In embodiments, a male luer lock 18 may be provided at the distal end of the outlet tube 16 and mated therewith. In embodiments, the male luer lock 18 may be connected to additional tubing and an intravenous needle or a catheter that may be inserted into or otherwise in fluid communication with a vein of the patient. As illustrated in FIG. 1, for example, the mixing element 14 may be provided at the distal end of the multiple line portion 12, which is positioned adjacent to or otherwise near the patient. In embodiments, mixing of the fluids provided by the lines 12a, 12b, 12c and 12d is provided at a point close to the patient which reduces the likelihood of delay in administration of the fluid and makes it less likely that a bolus of fluid will back up during the mixing process.

In embodiments, when fluid delivery via one of the lines 12a, 12b, 12c and 12d is complete, it may be desirable to clear remaining fluid in the line without flushing it into the patient's body. In such cases, the fluid may be drawn back up the line away from the user's body, however, this may also result in drawing blood from the user's body, which is undesirable. In embodiments, a one-way valve may be provided at a distal end of the outlet tube 16. In embodiments, the one-way valve may prevent the withdrawal of blood from the patient into the outlet tube 16 while allowing for withdrawal of fluid from the lines 12a, 12b, 12c and 12d. In embodiments, the one-way valve may be provided upstream of the male luer lock 18. In embodiments, the one-way valve may be provided downstream of the male luer lock 18. In embodiments, the one-way valve may be positioned between the mixing element 14 and the outlet tube 16.

In embodiments, as noted above, the individual lines 14a, 14b, 14c and 14d may be separated or separable at least along a portion of their length and connected to separate fluid supplies so that the system 10 may provide various intravenous fluids to a patient via one IV needle or catheter. In FIG. 1, the lines 12a, 12b, 12c and 12d are partially separated at a proximal end thereof but remain connected below the binding element 19. In embodiments, the lines 12a, 12b, 12c, 12d may be separated further or less, if desired. In embodiments, one or more of the lines 12b, 12c or 12d may be separated from the central line 12a while other lines may remain connected. In embodiments, the connection between the lines 12b, 12c, 12d and the line 12a is provided with a thickness that allows for easy separation of the lines 12b, 12c, 12d from the central line 12a. In embodiments, the thickness of the connection between the lines 12b, 12c, 12d and 12a may be set such that the lines are separable using a user's hands. In embodiments, the thickness of the connection between the lines 12b, 12c, 12d and the line 12a may be between 0.005 in. and 0.030 in. In embodiments, the thickness of the connection may vary depending on the material used. In embodiments, where PVC thermoplastic is used, the thickness may be 0.10 in. In embodiments, the thickness of the connection may be selected based on a balance of ease of manufacture, or ease of extruding, integrity of the connection and ease of separating the lines 12b, 12c, 12d and the line 12a. In embodiments, different thicknesses may be used.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. An intravenous fluid delivery system comprising:
a multiple line portion,
the multiple line portion including a plurality of fluid lines configured to transport fluid, wherein the plurality of fluid lines are connected to each other; and
a mixing element provided at a distal end of the multiple line portion wherein each fluid line of the plurality of fluid lines is in fluid communication with the mixing element, the mixing element including a single outlet in fluid communication with each fluid line of the plurality of fluid lines such that fluid from each of the fluid lines flows through the single outlet.

2. The intravenous fluid delivery system of claim 1, wherein the plurality of fluid lines includes a central line and one or more peripheral lines positioned around the central line.

3. The intravenous fluid delivery system of claim 2, where the one or more peripheral lines are connected to an external wall of the central line.

4. The intravenous fluid delivery system of claim 3, wherein the one or more peripheral lines are detachably connected to the central line along at least a portion of a length of the one or more peripheral lines and the central line.

5. The intravenous fluid delivery system of claim 3, wherein the one or more peripheral lines are separable from the central line along at least a portion of the one or more peripheral lines and the central line.

6. The intravenous fluid delivery system of claim 3, wherein a thickness of a connection between the central line and the one or more peripheral lines is between 0.005 in and 0.030 in.

7. The intravenous fluid delivery system of claim 1, wherein the plurality of fluid lines are made of PVC, silicone, Polyurethane or Tygon.

8. The intravenous fluid system of claim 1, wherein the plurality of fluid lines are extruded together.

9. The intravenous fluid system of claim 1, further comprising a plurality of connectors, wherein each connector of the plurality of connectors is configured for connection to a respective fluid source and a respective fluid line such that each of the fluid lines is connected to a respective fluid source.

10. The intravenous fluid system of claim 9, wherein the respective fluid source is an intravenous bag.

11. The intravenous system of claim 9, wherein the respective fluid source is an infusion pump.

12. The intravenous system of claim 1, wherein the mixing element includes a plurality of slots formed in an interior thereof, wherein the plurality of slots correspond to and are in fluid communication with the plurality of fluid lines.

13. The intravenous system of claim 2, wherein the mixing element includes a central slot corresponding to and in fluid communication with the central line and one or more peripheral slots corresponding to and in fluid communication with the one or more peripheral fluid lines.

14. The intravenous system of claim 13, wherein the one or more peripheral slots are configured to provide fluid to the central slot.

15. The intravenous system of claim 14, wherein the single outlet is formed by a distal end of the central slot such that fluid passing through the central slot, including fluid provided to the central slot from the one more peripheral slots passes through the single outlet.

16. The intravenous system of claim 1, further comprising an outlet tube configured to be received in and in fluid communication with the single outlet.

17. The intravenous system of claim 16, further comprising a second connector configured to connect the outlet tube to a catheter.

18. The intravenous system of claim 16, further comprising a second connector configured to connect the outlet tube to an intravenous needle.

19. The intravenous system of claim 16, further comprising a one-way valve provided downstream from the outlet tube and configured to prevent fluid from flowing upstream into the mixing element.

20. The intravenous system of claim 16, further comprising a one-way valve positioned between the outlet tube and the mixing element and configured to prevent fluid from flowing upstream into the mixing element.
